(19) United States
(12) Patent Application Publication
(10) Pub. No.: US 2022/0044816 A1
(43) Pub. Date: Feb. 10, 2022

(54) INFORMATION PROCESSING APPARATUS AND NON-TRANSITORY COMPUTER READABLE MEDIUM

(71) Applicant: FUJIFILM BUSINESS INNOVATION CORP., Tokyo (JP)

(72) Inventors: Hideto ODA, Kanagawa (JP); Kiyoshi IIDA, Kanagawa (JP); Noriji KATO, Kanagawa (JP)

(73) Assignee: FUJIFILM BUSINESS INNOVATION CORP., Tokyo (JP)

(21) Appl. No.: 17/167,181

(22) Filed: Feb. 4, 2021

(30) Foreign Application Priority Data
Aug. 4, 2020 (JP) 2020-132182

(51) Int. Cl. G16H 50/30; A61B 5/024; A61B 5/00; G16H 50/50

(52) U.S. Cl. CPC G16H 50/30; A61B 5/024; A61B 5/0245; A61B 5/7267; G16H 50/50; A61B 5/7246

(57) ABSTRACT

An information processing apparatus includes a processor configured to estimate a waveform of heartbeats by inputting a waveform of a measured pulse wave to a model constructed by mounting a pulse wave measurement device and a heartbeat measurement device to a test subject and calculating the relationship between respective waveforms output from the devices.

### CROSS-REFERENCE TO RELATED APPLICATIONS

This application is based on and claims priority under 35 USC 119 from Japanese Patent Application No. 2020-132182 filed Aug. 4, 2020.

### BACKGROUND

### (i) Technical Field

The present disclosure relates to an information processing apparatus and a non-transitory computer readable medium.

### (ii) Related Art

A hyperdynamic state of the autonomic nerves is observed by measuring the interval of heartbeats from an electrocardiogram. For example, it is observed that the autonomic nerves are tense if the interval of heartbeats is short, and that the autonomic nerves are relaxed if the interval of heartbeats is long.

An example of such a technique is described in Japanese Unexamined Patent Application Publication No. 2018-130319.

### SUMMARY

It is conceivable to measure the interval of heartbeats using the interval of pulses, since it is convenient etc. Pulses may be measured using a wristband-type device, for example. However, using the interval of pulses, as it is, as the interval of heartbeats may lower the precision in estimating the waveform of heartbeats because of the difference in properties between pulses and heartbeats.

Aspects of non-limiting embodiments of the present disclosure relate to enhancing the precision in estimating the waveform of heartbeats when measuring the interval of heartbeats using the interval of pulses, compared to the case where the interval of pulses, as it is, is used as the interval of heartbeats.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided an information processing apparatus including a processor configured to estimate a waveform of heartbeats by inputting a waveform of measured pulses to a model constructed by mounting a pulse wave measurement device and a heartbeat measurement device to a test subject and calculating a relationship between respective waveforms output from the devices.

### BRIEF DESCRIPTION OF THE DRAWINGS

Exemplary embodiments of the present disclosure will be described in detail based on the following figures, wherein:

FIG. 1 illustrates an overview of an apparatus system that is used in a first exemplary embodiment, in which (A) illustrates an example of the configuration of a learning system, and (B) illustrates an example of the configuration of an estimation system;

FIG. 2 illustrates a waveform that constitutes electrocardiographic waveform data;

FIGS. 3A and 3B illustrate an example of electrocardiographic waveform data, in which FIG. 3A illustrates electrocardiographic waveform data at the time when beats are slow, and FIG. 3B illustrates electrocardiographic waveform data at the time when beats are quick;

FIGS. 4A and 4B illustrate fluctuations in heartbeats, in which FIG. 4A is a tachogram that indicates fluctuations in the heartbeat interval, and FIG. 4B is a chart that indicates the power spectral density;

FIG. 5 illustrates the difference in the waveform, and the deviation in the peak position in the time direction, between electrocardiographic waveform data and pulse wave data, in which (A) illustrates an example of pulse wave data, and (B) illustrates an example of electrocardiographic waveform data;

FIG. 6A to 6C illustrate the difference in the waveform of pulse wave data due to the difference in the position of measurement, in which FIG. 6A indicates pulse wave data measured at a fingertip, FIG. 6B indicates pulse wave data measured at an earlobe, and FIG. 6C indicates pulse wave data measured at a wrist;

FIG. 7 illustrates an example of the hardware configuration of a model generation device that is used in the first exemplary embodiment;

FIG. 8 illustrates an example of the functional configuration of a model learning device that is used in the first exemplary embodiment;

FIG. 9 illustrates an example of the hardware configuration of a heartbeat estimation device that is used in the first exemplary embodiment;

FIG. 10 illustrates an example of the functional configuration of an electrocardiographic waveform estimation device that is used in the first exemplary embodiment;

FIG. 11 illustrates the deviation in the time direction between the heartbeat interval and the pulse interval;

FIG. 12 illustrates an example of the functional configuration of a model learning device that is used in a second exemplary embodiment;

FIG. 13 is a flowchart illustrating an example of processing operation executed by a time deviation correction section;

FIGS. 14A and 14B illustrate the effect of a correction of the deviation in the time, in which FIG. 14A indicates the correct answer rate of an autonomic nerve index before correcting the deviation in the time, and FIG. 14B indicates the correct answer rate of an autonomic nerve index after correcting the deviation in the time;

FIG. 15 illustrates abnormal values;

FIG. 16 illustrates an example of the functional configuration of a model learning device that is used in a third exemplary embodiment;

FIGS. 17A and 17B illustrate a quotient filter, in which FIG. 17A indicates an example of a value treated as a normal value, and FIG. 17B indicates a specific example;

FIG. 18 illustrates a different example of the position at which pulse wave data are measured, in which (A) illustrates an example of the configuration of a learning system, and (B) illustrates an example of the configuration of an estimation system; and

FIG. 19 illustrates a different example of the position at which pulse wave data are measured, in which (A) illustrates an example of the configuration of a learning system, and (B) illustrates an example of the configuration of an estimation system.

### DETAILED DESCRIPTION

Exemplary embodiments will be described in detail below with reference to the accompanying drawings.

### First Exemplary Embodiment

### <System Configuration>

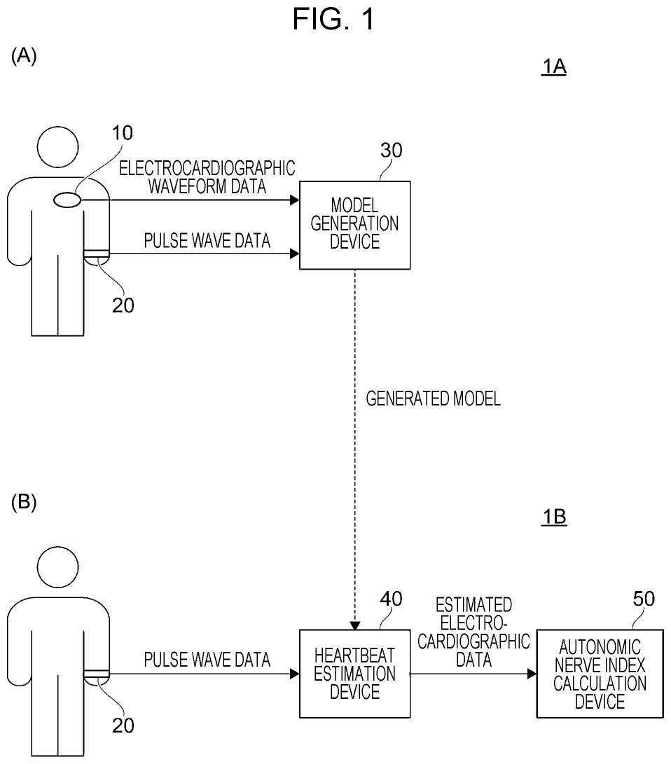

FIG. 1 illustrates an overview of an apparatus system that is used in a first exemplary embodiment. In FIG. 1, (A) illustrates an example of the configuration of a learning system 1A, and (B) illustrates an example of the configuration of an estimation system 1B.

The learning system 1A includes an electrocardiographic sensor 10 that measures an electric signal generated along with motion of a heart of a test subject, a pulse wave sensor 20 that measures the waveform of a pulse wave that appears at a fingertip of the test subject, and a model generation device 30 that learns the relationship between electrocardiographic waveform data and pulse wave data measured concurrently for the identical test subject.

The electrocardiographic waveform data are an example of heartbeat waveform data.

The electrocardiographic sensor 10 according to the present exemplary embodiment is a sensor that measures variations in the electric signal that accompany motion of the heart as electrocardiographic waveform data. The electrocardiographic sensor 10 includes a plurality of electrode pads to be mounted so as to interpose the heart, an amplifier that amplifies an electric signal generated in the electrode pads, an analog/digital conversion section that converts the amplified electric signal into a digital signal, and a computation section that generates electrocardiographic waveform data from the digital signal. The electrocardiographic sensor 10 is an example of a heartbeat measurement device.

The pulse wave sensor 20 according to the present exemplary embodiment is a sensor that measures variations in the blood flow volume that accompany motion of the heart as a pulse wave. In the case of the present exemplary embodiment, the pulse wave sensor 20 measures a pulse wave through a photoplethysmographic method.

The photoplethysmographic method includes a transmissive type in which variations in the blood flow volume are measured through the amount of variations in light that transmits the body, and a reflective type in which variations in the blood flow volume are measured through the amount of variations in light reflected within the living body.

The pulse wave sensor 20 illustrated in FIG. 1 may be either a transmissive type or a reflective type. The pulse wave sensor 20 outputs the measurement result as pulse wave data. The pulse wave sensor 20 is an example of a pulse wave measurement device.

The model generation device 30 learns the relationship between electrocardiographic waveform data and pulse wave data measured concurrently from an identical test subject, and generates a model that outputs, on the basis of measured pulse wave data, electrocardiographic waveform data that are highly likely to be measured concurrently with the pulse wave data. In other words, the model generation device 30 is a computer that learns the relationship between electrocardiographic waveform data and pulse wave data that are measured using different measurement methods and at different positions.

In FIG. 1, a model generated by the model generation device 30 is represented as a “generated model”. The generated model is provided from the model generation device 30 to a heartbeat estimation device 40. The generated model is an example of a model constructed by calculating the relationship between the waveform of electrocardiographic waveform data and the waveform of pulse wave data.

The model generation device 30 is a so-called machine learning device. The model generation device 30 according to the present exemplary embodiment generates a generated model that is peculiar to a test subject (i.e. peculiar to each user) using electrocardiographic waveform data and pulse wave data measured for the test subject. A general-purpose generated model may also be generated using electrocardiographic waveform data and pulse wave data measured concurrently for a plurality of test subjects.

The model generation device 30 may acquire electrocardiographic waveform data and pulse wave data measured from an identical test subject by way of a local area network (LAN) or the Internet, or may acquire electrocardiographic waveform data and pulse wave data measured from an identical test subject from a database, a semiconductor memory, etc. (not illustrated).

The model generation device 30 may be constituted as a dedicated device that specializes in generating a generated model, or may be constituted as a server.

The estimation system 1B includes a pulse wave sensor 20 that measures the waveform of a pulse wave that appears at a fingertip of the test subject, a heartbeat estimation device 40 that estimates electrocardiographic waveform data from pulse wave data output from the pulse wave sensor 20 and outputs the electrocardiographic waveform data, and an autonomic nerve index calculation device 50 that calculates an autonomic nerve index by processing the estimated electrocardiographic waveform data (hereinafter also referred to as “estimated electrocardiographic data”).

The heartbeat estimation device 40 according to the present exemplary embodiment estimates, when pulse wave data output from the pulse wave sensor 20 are provided to the generated model, electrocardiographic waveform data (hereinafter referred to as “estimated electrocardiographic data”) that are highly likely to be measured from an identical test subject concurrently with the pulse wave data, and outputs the estimated electrocardiographic data.

The generated model used by the heartbeat estimation device 40 for estimation has been provided in advance from the model generation device 30. The heartbeat estimation device 40 is an example of an information processing apparatus.

The heartbeat estimation device 40 may acquire pulse wave data by way of a local area network (LAN) or the Internet, or may acquire pulse wave data from a database, a semiconductor memory, etc. (not illustrated).

The heartbeat estimation device 40 may be constituted as a dedicated device that estimates estimated electrocardiographic data from pulse wave data, may be constituted as a server, or may be constituted as a wearable terminal.

In FIG. 1, the heartbeat estimation device 40 is illustrated as a device that is independent of the pulse wave sensor 20. However, the heartbeat estimation device 40 may be constituted integrally with the pulse wave sensor 20.

The autonomic nerve index calculation device 50 according to the present exemplary embodiment is a device that calculates an autonomic nerve index given by the following formula, by performing a frequency analysis on time variations in the interval of heartbeats acquired from the estimated electrocardiographic data.

Autonomic nerve index=LF/HF Formula 1

LF is the power spectral density of an intermediate frequency component of the time variations in the interval of heartbeats. HF is the power spectral density of a high frequency component of the time variations in the interval of heartbeats.

The autonomic nerve index is also referred to as a stress index, and represents the degree of activity of the sympathetic nerves. In a relaxed state, the value of the autonomic nerve index is small. In a stressed state, the value of the autonomic nerve index is large.

In FIG. 1, the autonomic nerve index calculation device 50 is illustrated as a device that is independent of the heartbeat estimation device 40. However, the autonomic nerve index calculation device 50 may be constituted integrally with the heartbeat estimation device 40.

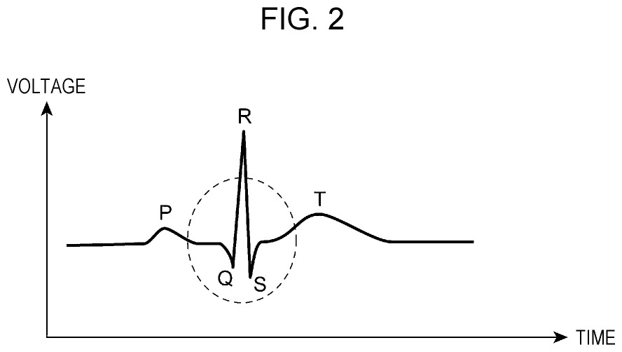

The terms to be used in relation to the present exemplary embodiment will be described below with reference to FIGS. 2 to 6A to 6C.

FIG. 2 illustrates a waveform that constitutes electrocardiographic waveform data. In FIG. 2, the horizontal axis represents the time, and the vertical axis represents the voltage. The waveform indicated in FIG. 2 represents an electric signal corresponding to one heartbeat.

The waveform indicated in FIG. 2 includes a P wave, Q, R, and S waves, and a T wave in the chronological order. The P wave is a waveform that appears when the atria are excited. The Q, R, and S waves are waveforms that appear when the ventricles are excited. The T wave is a waveform that appears when the ventricles are recovered from the excited state.

The R wave gives the peak position of the entire electric signal. In the present exemplary embodiment, the period from a certain R wave to the next R wave is referred to as the interval of heartbeats or the heartbeat interval.

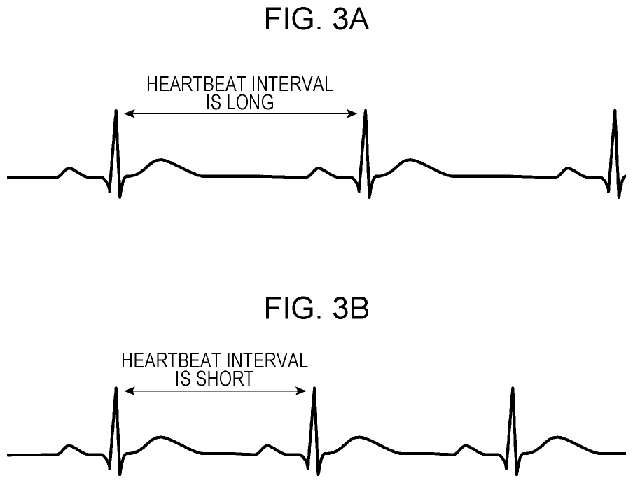

FIGS. 3A and 3B illustrate an example of electrocardiographic waveform data. FIG. 3A illustrates electrocardiographic waveform data at the time when beats are slow. FIG. 3B illustrates electrocardiographic waveform data at the time when beats are quick.

In the case where the heartbeat interval is long, the parasympathetic nerves are in a hyperdynamic state. This state appears in a relaxed state.

In the case where the heartbeat interval is short, the sympathetic nerves are in a hyperdynamic state. This state appears in a tense state.

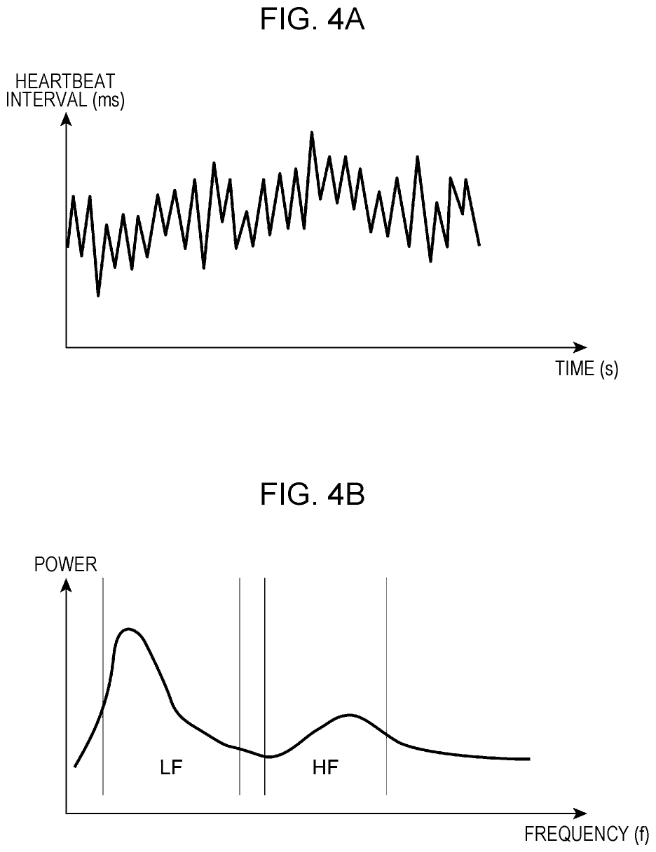

FIGS. 4A and 4B illustrate fluctuations in heartbeats. FIG. 4A is a tachogram that indicates fluctuations in the heartbeat interval. FIG. 4B is a chart that indicates the power spectral density.

In FIG. 4A which is a tachogram indicating heartbeats, the horizontal axis represents the time, and the vertical axis represents the heartbeat interval.

In FIG. 4B which indicates the power spectral density, the horizontal axis represents the frequency, and the vertical axis represents the power. The frequency domain of LF which is an intermediate frequency component is given as 0.04 to 0.15 Hz, for example. The frequency domain of HF which is a high frequency component is given as 0.16 to 0.40 Hz, for example. The power spectral density is calculated as the value of power per unit frequency width (i.e. a width of 1 Hz).

The autonomic nerve index calculation device 50 (see FIG. 1) discussed earlier calculates an autonomic nerve index as the ratio between the total value (i.e. an integrated value) of power in the frequency domain of the LF component and the total value of power in the frequency domain of the HF component, for example.

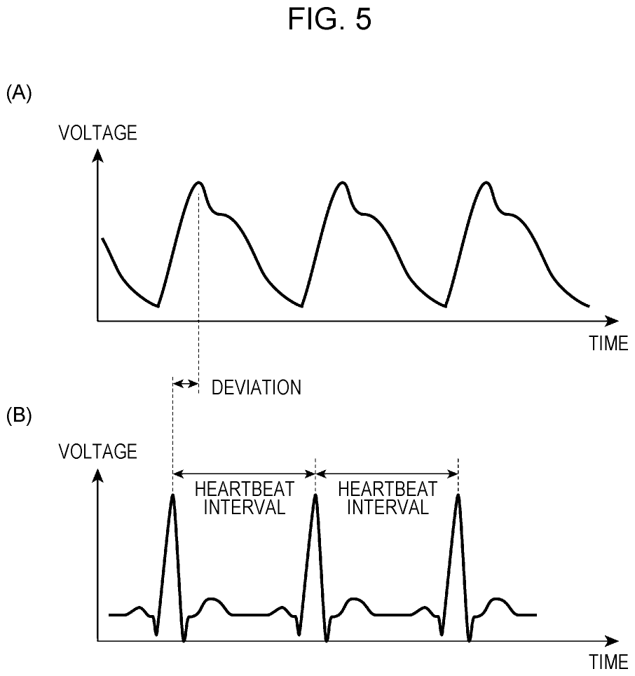

FIG. 5 illustrates the difference in the waveform, and the deviation in the peak position in the time direction, between electrocardiographic waveform data and pulse wave data. In FIG. 5, (A) illustrates an example of pulse wave data, and (B) illustrates an example of electrocardiographic waveform data.

As indicated in FIG. 5, pulse wave data measured through a photoplethysmographic method have a gently undulating waveform. On the other hand, electrocardiographic waveform data have a pulsed waveform. Because of the difference in the waveform, the precision in specifying peak positions from the pulse wave data is low compared to the precision in specifying peak positions from the electrocardiographic waveform data. In other words, the precision in the interval of pulses calculated from the pulse wave data is low compared to the precision in the interval of heartbeats calculated from the electrocardiographic waveform data.

The peak positions in the pulse wave data tend to be delayed compared to the peak positions in the electrocardiographic waveform data. That is, there tends to be a deviation in the time axis direction.

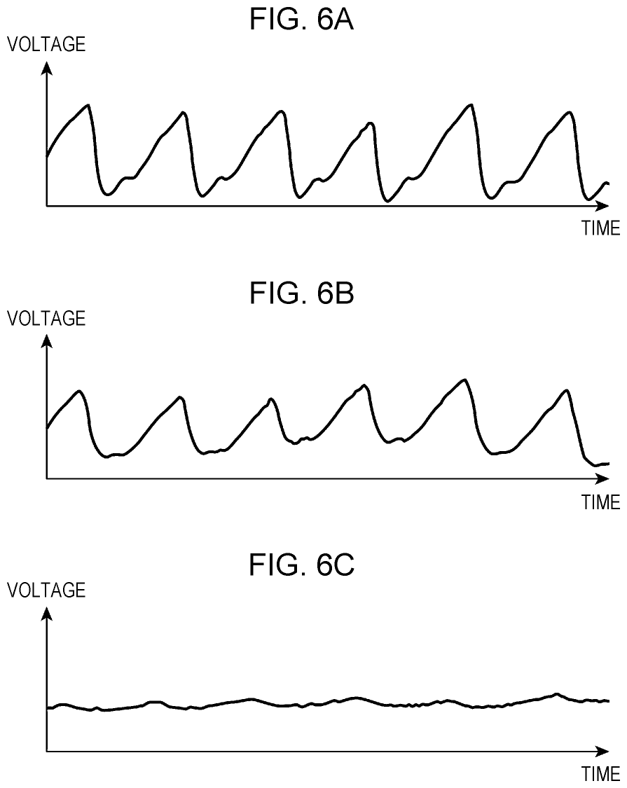

FIG. 6A to 6C illustrate the difference in the waveform of pulse wave data due to the difference in the position of measurement (hereinafter also referred to as a “location of measurement”). FIG. 6A indicates pulse wave data measured at a fingertip. FIG. 6B indicates pulse wave data measured at an earlobe. FIG. 6C indicates pulse wave data measured at a wrist.

If the density of capillary vessels is different, the signal level of measured pulse wave data is also varied. In the case of FIGS. 6A to 6C, the signal level of pulse wave data measured at a fingertip is the highest, the signal level of pulse wave data measured at an earlobe is the second highest, and the signal level of pulse wave data measured at a wrist is the lowest. In the case of FIGS. 6A to 6C, variations in the signal level of pulse wave data measured at a wrist are small. Therefore, the precision in detecting peak positions measured at a wrist is low compared to the precision in detecting peak positions measured at the two other positions.

The density of capillary vessels is different not only among positions of measurement but also among test subjects.

Body motion of the test subject varies the blood flow volume. Such variations are superimposed on pulse wave data as noise (hereinafter also referred to as “body motion noise”). The body motion noise affects pulse wave data measured at a wrist particularly significantly compared to data measured at other locations.

In this manner, the waveform of pulse wave data may differ in the waveform because of the difference in the measurement method, may differ in the signal level because of the difference in the measurement position, and may be affected by body motion noise.

Therefore, there is a low correlation between the interval of pulses (hereinafter referred to as a “pulse interval”) simply calculated from pulse wave data and the heartbeat interval specified from electrocardiographic waveform data.

<Configuration of Device>

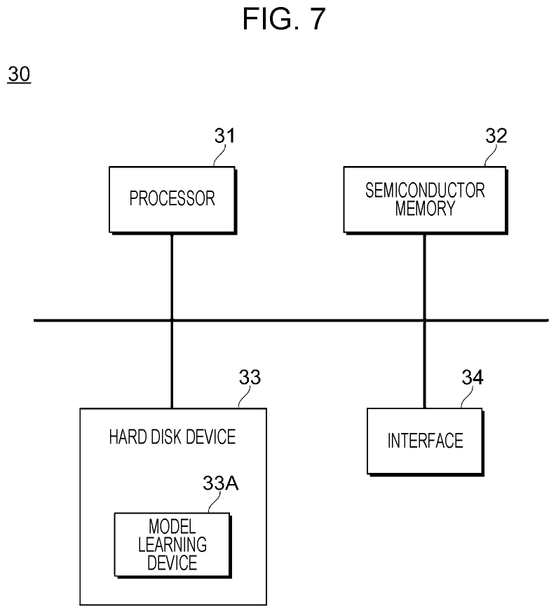

FIG. 7 illustrates an example of the hardware configuration of the model generation device 30 that is used in the first exemplary embodiment.

The model generation device 30 includes a processor 31 that processes data, a semiconductor memory 32 that serves as a principal storage device, a hard disk device 33 that serves as an auxiliary storage device, and an interface 34 that transmits and receives data to and from an external device. The processor 31 and the other components are connected to each other through a bus or a signal line.

The processor 31 is a central processing unit (CPU), for example. The semiconductor memory 32 includes a read only memory (ROM) that stores a basic input output system (BIOS) etc. and a random access memory (RAM) that is used as a work area.

The hard disk device 33 is a storage device that stores basic software and application programs (hereinafter referred to as “apps”). The hard disk device 33 may be a non-volatile semiconductor memory.

In the case of the present exemplary embodiment, a model learning device 33A that learns the relationship between electrocardiographic waveform data and pulse wave data is stored as an example of the apps.

The model learning device 33A learns such that estimated electrocardiographic data estimated from input pulse wave data coincide with electrocardiographic waveform data measured from an identical test subject concurrently with the pulse wave data.

The interface 34 transmits and receives data to and from an external device using the Universal Serial Bus (USB) standard or the Local Area Network (LAN) standard, for example.

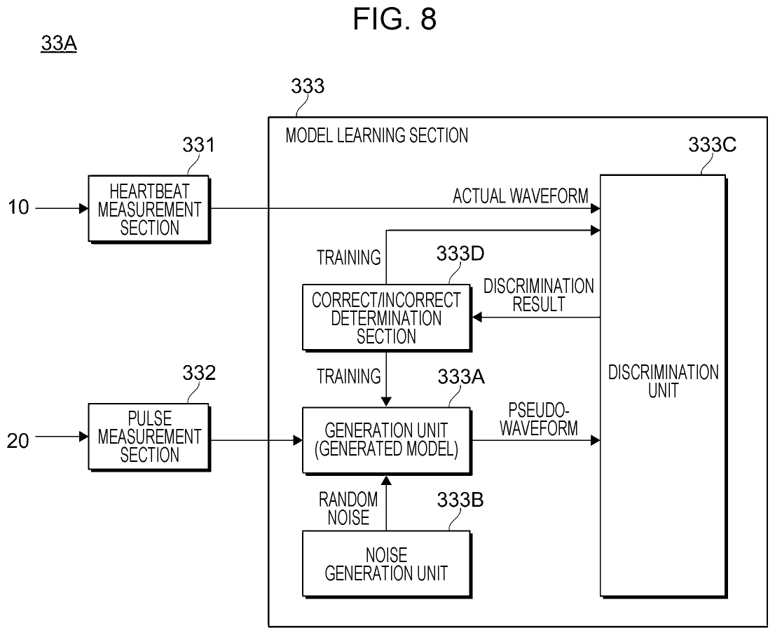

FIG. 8 illustrates an example of the functional configuration of the model learning device 33A that is used in the first exemplary embodiment.

The model learning device 33A illustrated in FIG. 8 includes a heartbeat measurement section 331, a pulse measurement section 332, and a model learning section 333.

The heartbeat measurement section 331 receives electrocardiographic waveform data from the electrocardiographic sensor 10, and measures the interval of heartbeats. Specifically, the heartbeat measurement section 331 determines the interval of heartbeats by calculating the time difference between the time of occurrence of an R wave (see FIG. 2) detected from the electrocardiographic waveform data and the time of occurrence of the preceding R wave. The interval of heartbeats is referred to as an R-R Interval (RRI).

The pulse measurement section 332 receives pulse wave data from the pulse wave sensor 20, and measures the interval of adjacent peak points. Specifically, the pulse measurement section 332 determines the pulse interval by calculating the time difference between the time of occurrence of a peak point detected from the pulse wave data and the time of occurrence of the preceding peak point. The pulse interval is referred to as an Inter Beat Interval (IBI).

The model learning section 333 learns the relationship between the electrocardiographic waveform data and the pulse wave data, and generates, from the pulses, a waveform from which the difference between the heartbeats and the pulses due to the difference in the method of measurement or the position of measurement has been excluded.

The model learning section 333 includes a generation unit 333A that generates, from the pulse wave data, a waveform (hereinafter referred to as a “pseudo-waveform”) that may be erroneously recognized as genuine by a discrimination unit 333C, a noise generation unit 333B that generates random noise, the discrimination unit 333C which determines whether each of the pseudo-waveform generated by the generation unit 333A and the electrocardiographic waveform data (hereinafter also referred to as an “actual waveform”) provided from the heartbeat measurement section 331 is genuine or false, and a correct/incorrect determination section 333D that determines whether or not the result (hereinafter referred to as a “discrimination result”) of the discrimination by the discrimination unit 333C is correct.

The generation unit 333A generates a pseudo-waveform on the basis of the pulse wave data and random noise. The generation unit 333A learns a relationship that generates a pseudo-waveform that may be erroneously discriminated as an actual waveform on the basis of feedback (hereinafter referred to as “training”) from the correct/incorrect determination section 333D.

This learning uses a Least Squares Generative Adversarial Network (LSGAN) which is an example of conditional Generative Adversarial Networks (GANs). The LSGAN is an example of unsupervised learning.

The generation unit 333A which has learned is transplanted to the heartbeat estimation device 40 (see FIG. 1) as a generated model.

The discrimination unit 333C alternately receives an actual waveform and a pseudo-waveform. The pseudo-waveform is also input to the discrimination unit 333C as a genuine waveform. The discrimination unit 333C discriminates whether each input waveform is genuine or false. The discrimination unit 333C learns, on the basis of training from the correct/incorrect determination section 333D, so as not to erroneously discriminate a pseudo-waveform as a genuine waveform.

As the precision in the discrimination by the discrimination unit 333C becomes higher, the pseudo-waveform generated by the generation unit 333A also becomes more similar to the actual waveform. The discrimination unit 333C outputs the result of discriminating whether the input pseudo-waveform is genuine or false to the correct/incorrect determination section 333D.

The correct/incorrect determination section 333D determines whether or not the result of the discrimination by the discrimination unit 333C is correct, and feeds back the determination result to the generation unit 333A and the discrimination unit 333C. This feedback is referred to as “backward propagation of errors”.

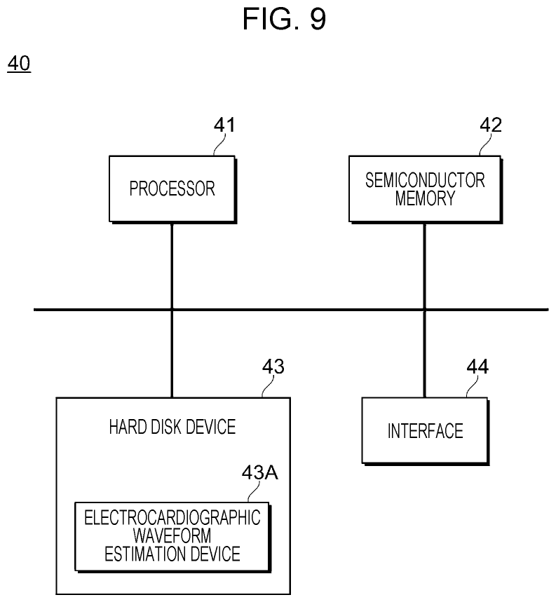

FIG. 9 illustrates an example of the hardware configuration of the heartbeat estimation device 40 that is used in the first exemplary embodiment.

The heartbeat estimation device 40 includes a processor 41 that processes data, a semiconductor memory 42 that serves as a principal storage device, a hard disk device 43 that serves as an auxiliary storage device, and an interface 44 that transmits and receives data to and from an external device. The processor 41 and the other components are connected to each other through a bus or a signal line.

The processor 41 is a CPU, for example. The semiconductor memory 32 includes a ROM that stores a BIOS etc., and a RAM that is used as a work area.

The hard disk device 43 is a storage device that stores basic software and apps. The hard disk device 43 may be a non-volatile semiconductor memory.

In the case of the present exemplary embodiment, an electrocardiographic waveform estimation device 43A that generates estimated electrocardiographic data from pulse wave data and outputs the estimated electrocardiographic data is stored as an example of the apps.

The electrocardiographic waveform estimation device 43A estimates, from the input pulse wave data, heartbeat waveform data that are highly likely to be measured concurrently, and outputs the estimated electrocardiographic waveform data as the estimated electrocardiographic data.

The interface 44 transmits and receives data to and from an external device using the USB standard or the LAN standard, for example.

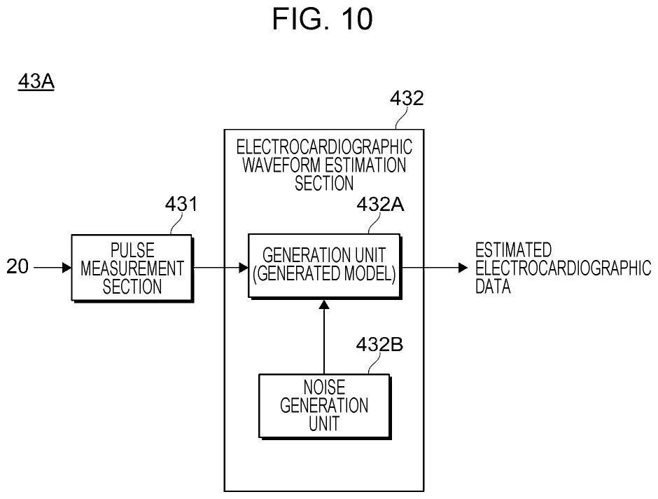

FIG. 10 illustrates an example of the functional configuration of the electrocardiographic waveform estimation device 43A that is used in the first exemplary embodiment.

The electrocardiographic waveform estimation device 43A illustrated in FIG. 10 includes a pulse measurement section 431 and an electrocardiographic waveform estimation section 432.

The pulse measurement section 431 receives pulse wave data from the pulse wave sensor 20, and measures the interval of adjacent peak points. The pulse measurement section 431 is the same as the pulse measurement section 332 (see FIG. 8).

The electrocardiographic waveform estimation section 432 includes a generation unit 432A that generates estimated electrocardiographic data from pulse wave data and a noise generation unit 432B that generates random noise.

The generated model generated by the model generation device 30 (see FIG. 1) is used as the generation unit 432A. That is, the generation unit 432A is identical to the generation unit 333A (see FIG. 8). The generation unit 432A estimates electrocardiographic waveform data that are highly likely to be measured concurrently with the input pulse wave data, and outputs the estimated electrocardiographic waveform data as the estimated electrocardiographic data.

### <Conclusion>

As discussed earlier, the learning system 1A (see FIG. 1) receives pulse wave data and electrocardiographic waveform data measured concurrently from an identical test subject, and learns the relationship between the two waveforms so as to generate, from input pulse wave data, electrocardiographic waveform data corresponding thereto.

On the other hand, the estimation system 1B, to which a generated model generated by the generation unit 333A (see FIG. 8) which has learned has been transplanted, generates estimated electrocardiographic data, which are difficult to tell whether correct or not, from pulse wave data, for which both the method of measurement and the position of measurement are different from those for electrocardiographic waveform data.

### Second Exemplary Embodiment

In the present exemplary embodiment, the precision of estimation is improved compared to the first exemplary embodiment.

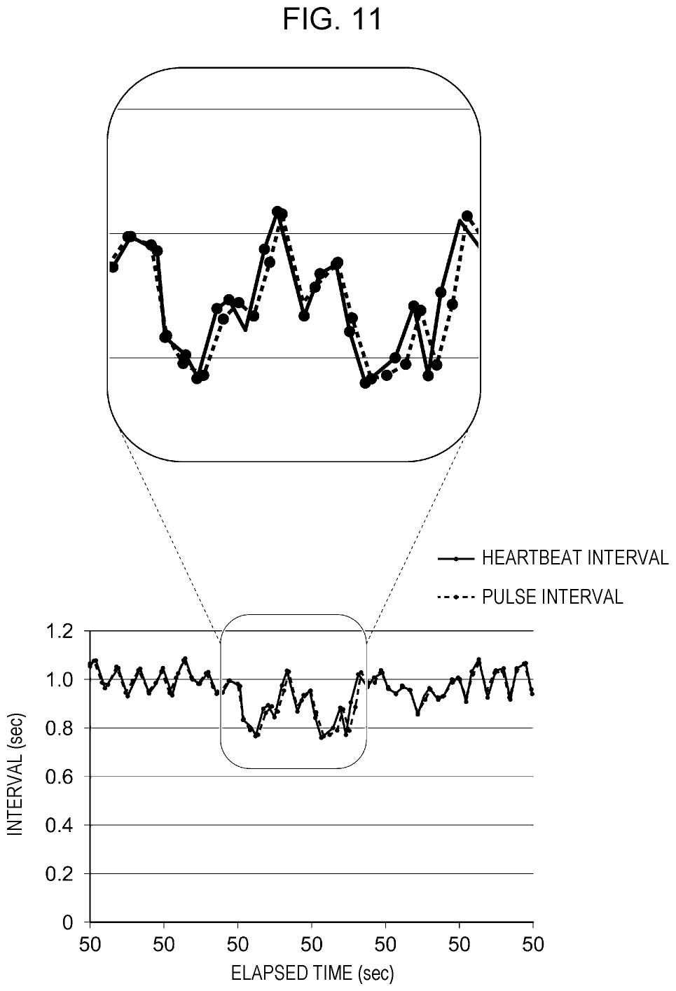

FIG. 11 illustrates the deviation in the time direction between the heartbeat interval and the pulse interval. In FIG. 11, the horizontal axis represents the time, and the vertical axis represents the interval. The interval on the vertical axis is represented in units of milliseconds. The interval on the vertical axis corresponds to the heartbeat interval and the pulse interval.

In FIG. 11, a portion in which there is a deviation between the heartbeat interval and the pulse interval is illustrated as enlarged. In the enlarged portion, it is indicated that the pulse interval is delayed with respect to the heartbeat interval. In practice, there is occasionally caused a deviation (hereinafter also referred to as a “delay”), in the time direction, of the pulse interval compared to the heartbeat interval.

The deviation in the time direction indicated in FIG. 11 is difficult to detect using a technique of detecting an abnormal value. In the present exemplary embodiment, a processing section to remove such a deviation is added.

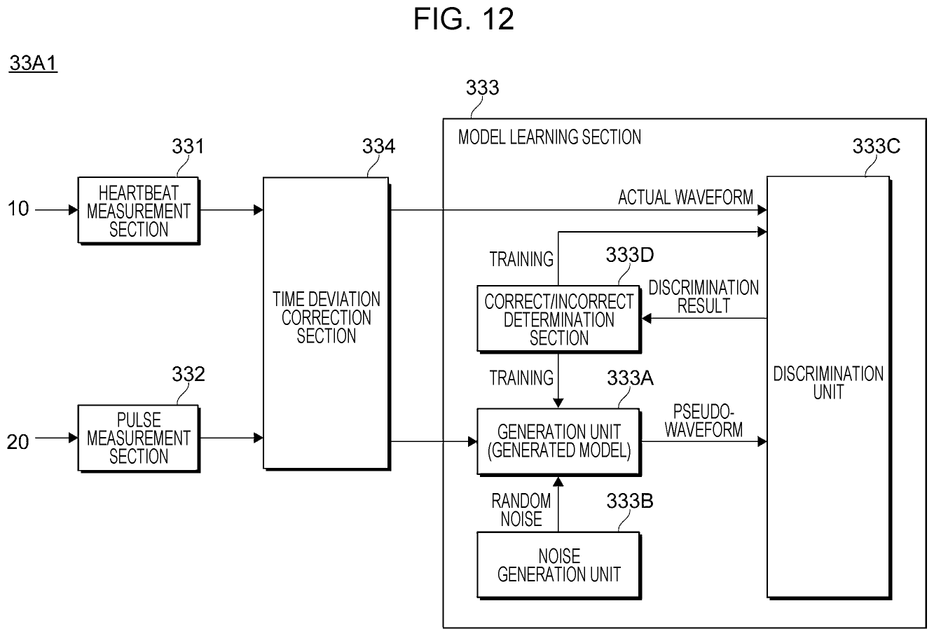

FIG. 12 illustrates an example of the functional configuration of a model learning device 33A1 that is used in a second exemplary embodiment. Portions in FIG. 12 corresponding to those in FIG. 8 are denoted by the corresponding reference numerals.

The model learning device 33A1 illustrated in FIG. 12 differs from the model learning device 33A illustrated in FIG. 8 in that a time deviation correction section 334 is provided before the model learning section 333. The time deviation correction section 334 illustrated in FIG. 12 receives electrocardiographic waveform data and pulse wave data, and delays an advanced one of the two data so as to match the other which is delayed. Consequently, learning by the generation unit 333A progresses with the electrocardiographic waveform data and the pulse wave data in phase with each other.

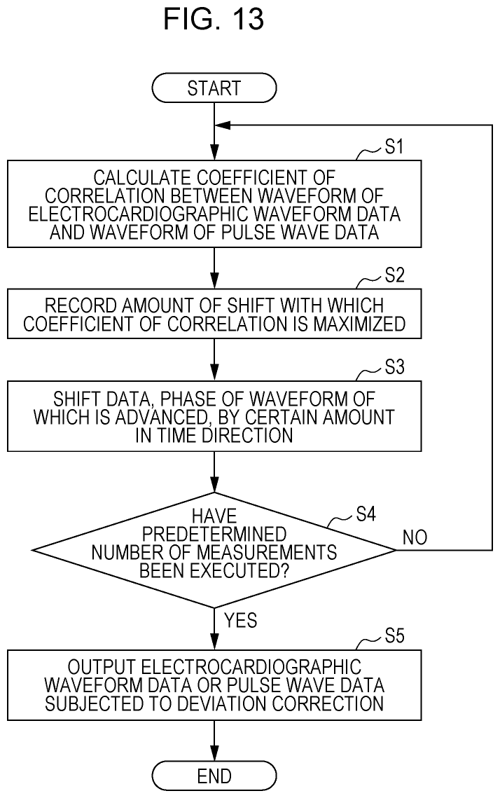

FIG. 13 is a flowchart illustrating an example of processing operation executed by the time deviation correction section 334. The symbol “S” in the drawing signifies a step.

First, the time deviation correction section 334 calculates a coefficient of correlation between the waveform of electrocardiographic waveform data and the waveform of pulse wave data (step 1).

Next, the time deviation correction section 334 records the amount of shift with which the coefficient of correlation is maximized (step 2).

Subsequently, the time deviation correction section 334 shifts data, the phase of the waveform of which is advanced, by a certain amount (step 3). In the case where the pulse wave data are delayed compared to the electrocardiographic waveform data, for example, the electrocardiographic waveform data are delayed by a certain amount. In the case where the electrocardiographic waveform data are delayed compared to the pulse wave data, on the other hand, the pulse wave data are delayed by a certain amount.

After that, the time deviation correction section 334 determines whether or not a predetermined number of measurements have been executed (step 4).

In the case where a negative result is obtained in step 4, the time deviation correction section 334 returns to step 1, and repeatedly performs the processes in steps 1 to 3.

In the case where a positive result is obtained in step 4, on the other hand, the time deviation correction section 334 outputs the electrocardiographic waveform data or the pulse wave data which have been subjected to a deviation correction (step 5).

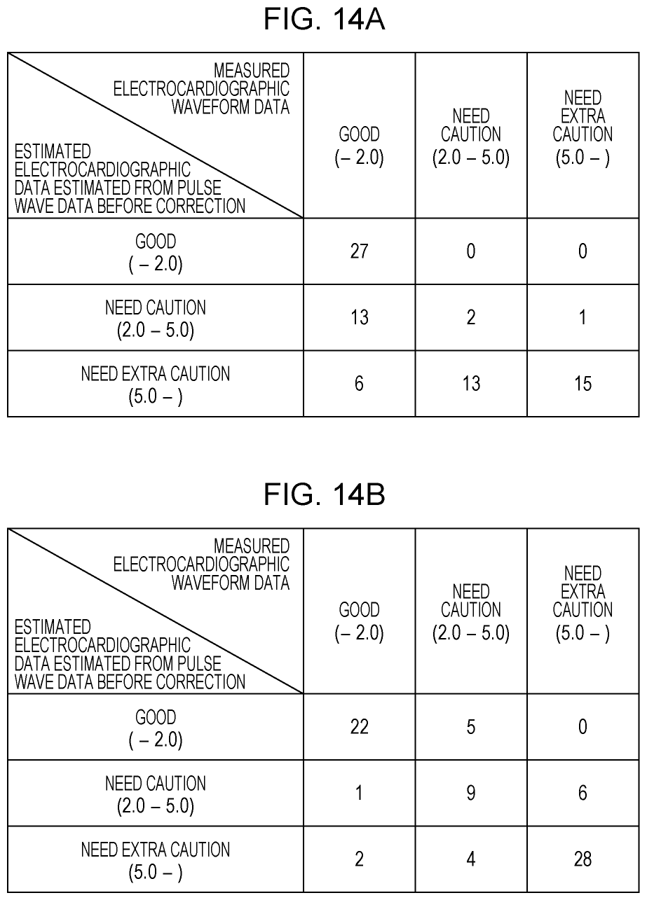

FIGS. 14A and 14B illustrate the effect of the correction of the deviation in the time. FIG. 14A indicates the correct answer rate of an autonomic nerve index before correcting the deviation in the time. FIG. 14B indicates the correct answer rate of an autonomic nerve index after correcting the deviation in the time.

The tables indicated in FIGS. 14A and 14B are each a confusion matrix that represents the relationship between divisions of the autonomic nerve index obtained for the measured electrocardiographic waveform data and divisions of the autonomic nerve index obtained for the estimated electrocardiographic data estimated from the pulse wave data.

The data indicated in FIGS. 14A and 14B are the result of processing measurement data for three test subjects for five days. The specific measurement time is 55.2 hours. The measurement data are divided in units of 30 seconds.

Six hundred and eight measurement data for 5.1 hours are used for learning by the generation unit 333A (see FIG. 8). Meanwhile, seventy seven measurement data for 0.6 hours which are not used for learning are used to estimate estimated electrocardiographic data. Apparently abnormal values are excluded.

In FIG. 14, the calculated numerical values of the autonomic nerve index are divided into three groups, namely “good”, “need caution”, and “need extra caution”, using two thresholds.

Then, in the example before a correction of the deviation in the time, the proportion (i.e. correct answer rate) at which the divisions of the autonomic nerve index calculated from the estimated electrocardiographic data coincide with the divisions of the autonomic nerve index calculated from the measured electrocardiographic waveform data is 57% (=44/77).

In the example after a correction of the deviation in the time, on the other hand, the proportion (i.e. correct answer rate) at which the divisions of the autonomic nerve index calculated from the estimated electrocardiographic data coincide with the divisions of the autonomic nerve index calculated from the measured electrocardiographic waveform data is 76% (=59/77).

### Third Exemplary Embodiment

Basically, no abrupt variations occur in the heartbeat interval or the pulse interval. However, abrupt variations occasionally appear in the actual measurement data. In many cases, such variations are caused by noise.

In the present exemplary embodiment, data varied abruptly compared to adjacent data are referred to as abnormal values, and are not used for learning.

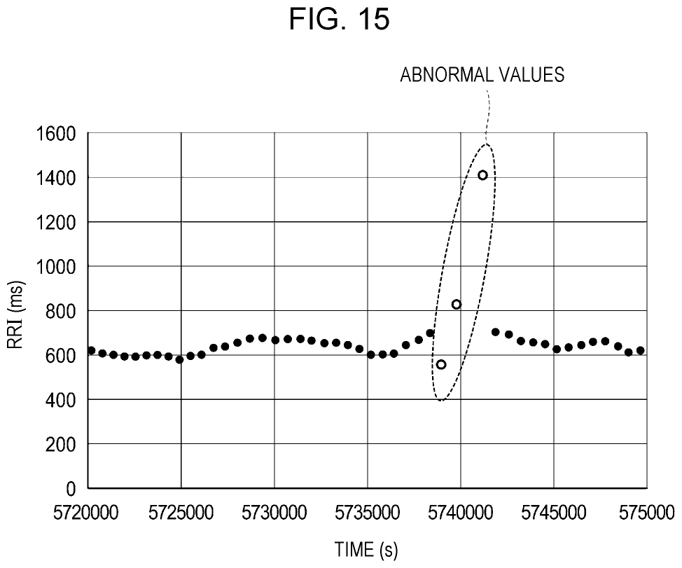

FIG. 15 illustrates abnormal values. In FIG. 15, the horizontal axis represents the time, and the vertical axis represents the interval. The interval corresponds to the heartbeat interval or the pulse interval. That is, the interval corresponds to the interval of peaks in the waveform of electrocardiographic waveform data or the interval of peaks in the waveform of pulse wave data.

Abnormal values frequently appear in data on the pulse interval compared to the heartbeat interval.

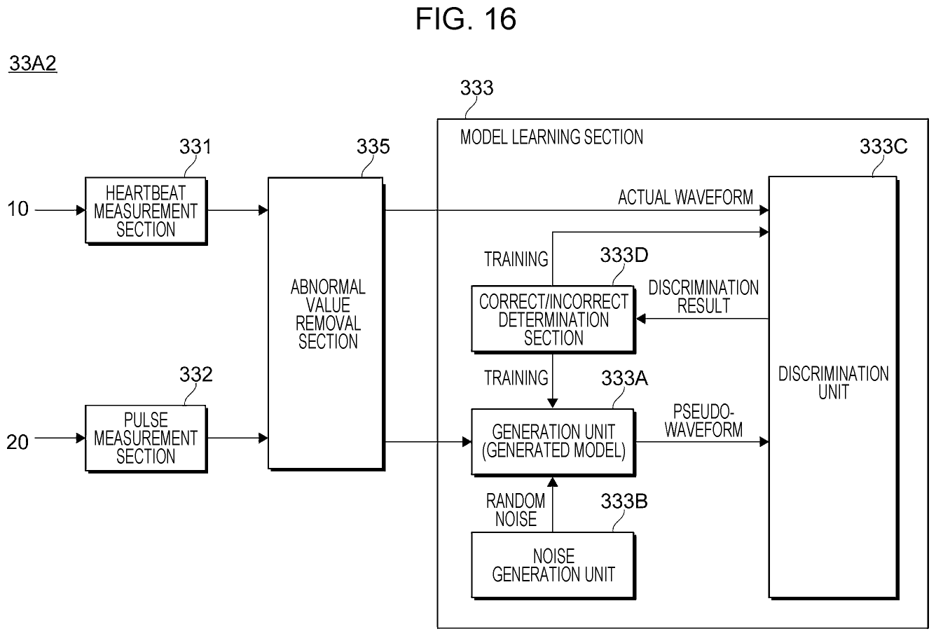

FIG. 16 illustrates an example of the functional configuration of a model learning device 33A2 that is used in a third exemplary embodiment. Portions in FIG. 16 corresponding to those in FIG. 8 are denoted by the corresponding reference numerals.

The model learning device 33A2 illustrated in FIG. 16 differs from the model learning device 33A illustrated in FIG. 8 in that an abnormal value removal section 335 is provided before the model learning section 333. The abnormal value removal section 335 illustrated in FIG. 15 receives electrocardiographic waveform data and pulse wave data, and removes detected abnormal values. Consequently, learning by the generation unit 333A progresses using electrocardiographic waveform data and pulse wave data including no abnormal values.

In the case of the present exemplary embodiment, the abnormal value removal section 335 uses a quotient filter.

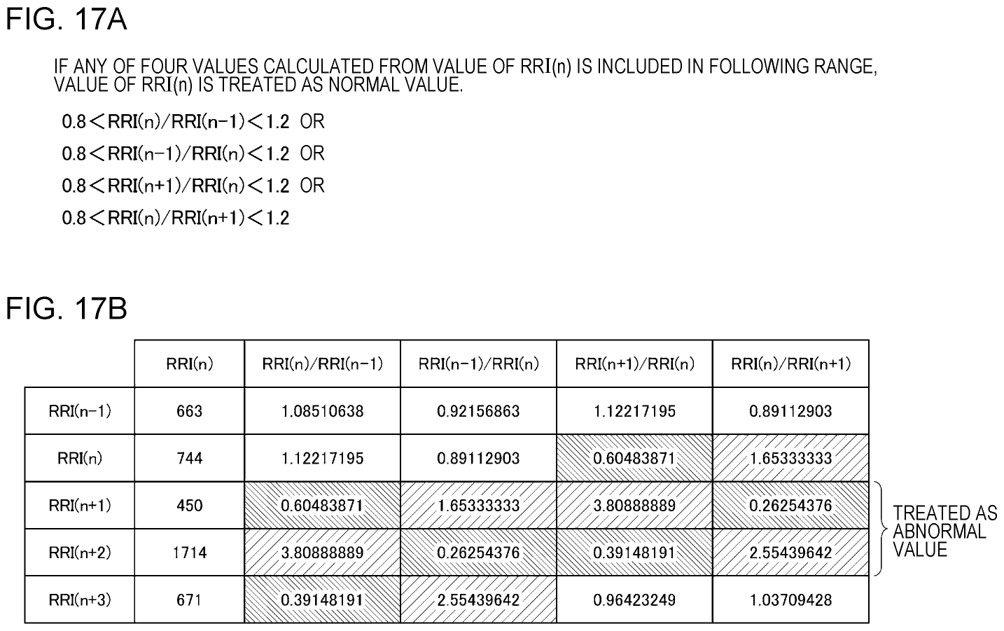

FIGS. 17A and 17B illustrate a quotient filter. FIG. 17A indicates an example of a value treated as a normal value. FIG. 17B indicates a specific example.

In the present exemplary embodiment, four quotients are calculated using the heartbeat interval at time n and two heartbeat intervals measured at time n−1 and n+1 which are before and after time n with different numerator/denominator relationships, and the corresponding heartbeat interval is determined as a normal value in the case where at least one of the four quotients is more than 0.8 and less than 1.2, and the corresponding heartbeat interval is determined as an abnormal value in the case where all the quotients are 0.8 or less or 1.2 or more, for example.

Therefore, in the example in FIG. 17, two values, namely RRI (n+1) and RRI (n+2) are determined as abnormal values. Similar determinations are also made for pulse intervals.

The abnormal value removal section 335 according to the present exemplary embodiment excludes data determined as an abnormal value from the target for learning. The abnormal value removal section 335 may inform the model learning section 333 of the range of normal values.

The abnormal value removal section 335 described in relation to the present exemplary embodiment may be combined with the time deviation correction section 334 (see FIG. 12) described in relation to the second exemplary embodiment.

Specifically, the abnormal value removal section 335 may be disposed before the time deviation correction section 334.

### Other Exemplary Embodiments

While exemplary embodiments of the present disclosure have been described above, the technical scope of the present disclosure is not limited to the exemplary embodiments discussed earlier. It is apparent from the following claims that a variety of modifications and improvements that may be made to the exemplary embodiments discussed earlier also fall within the technical scope of the present disclosure.

(1) In the exemplary embodiments discussed earlier, pulse wave data are measured at a fingertip. However, the position of measurement of pulse wave data is not limited to a fingertip.

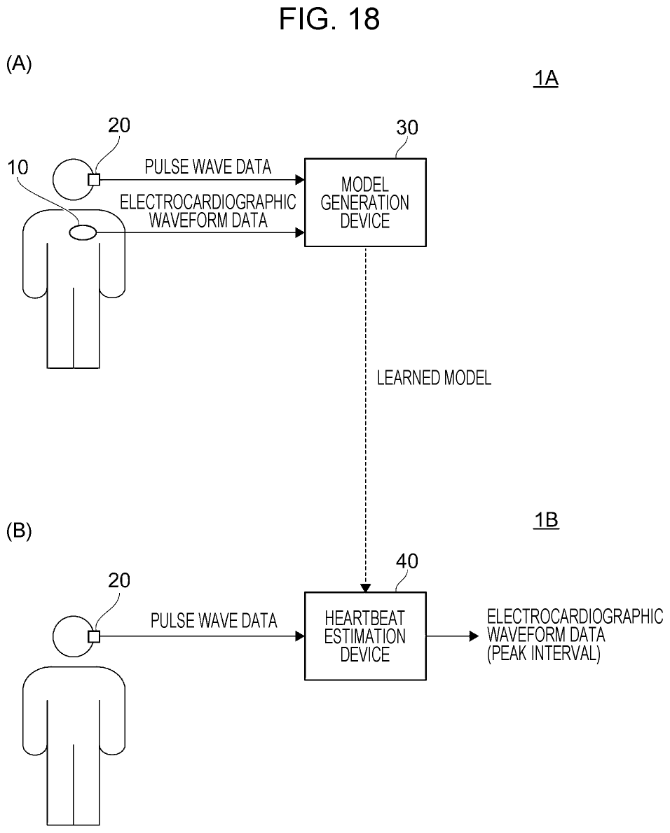

FIG. 18 illustrates a different example of the position at which pulse wave data are measured. In FIG. 18, (A) illustrates an example of the configuration of a learning system 1A, and (B) illustrates an example of the configuration of an estimation system 1B. Portions in FIG. 18 corresponding to those in FIG. 1 are denoted by the corresponding reference numerals. In the case of FIG. 18, pulse wave data are measured at an earlobe. In this case, the model generation device 30 learns the relationship between pulse wave data measured at an earlobe and electrocardiographic waveform data.

The position of measurement of pulse wave data is not limited to a fingertip and an earlobe, and may be a wrist or an ankle, or may be other positions of a human body.

(2) In the exemplary embodiments discussed earlier, a single generated model is learned at a time. However, a plurality of generated models may be learned at a time.

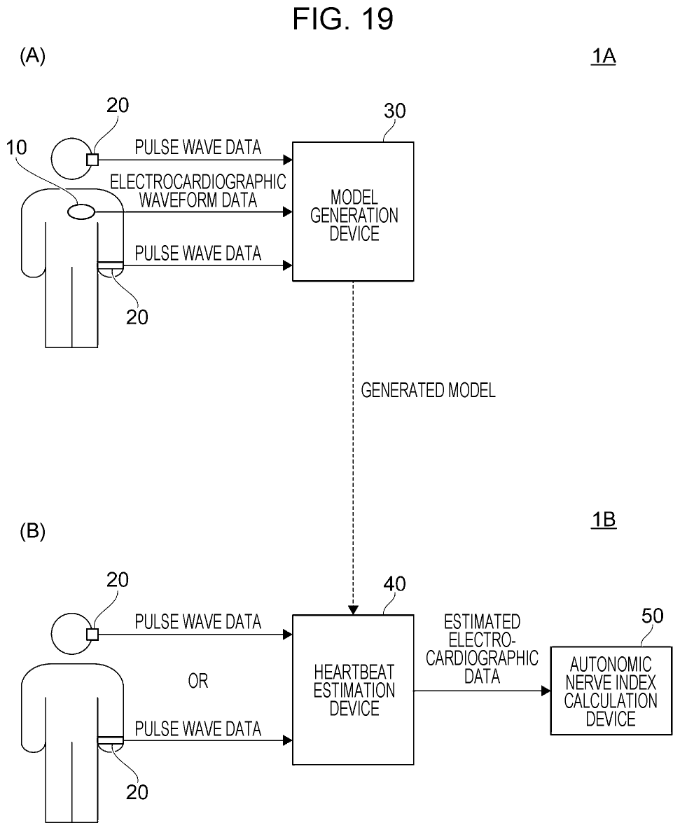

FIG. 19 illustrates a different example of the position at which pulse wave data are measured. In FIG. 19, (A) illustrates an example of the configuration of a learning system 1A, and (B) illustrates an example of the configuration of an estimation system 1B. Portions in FIG. 19 corresponding to those in FIG. 1 are denoted by the corresponding reference numerals.

In the example illustrated in FIG. 19, two generated models, namely a generated model that has learned the relationship between pulse wave data measured at a fingertip and electrocardiographic waveform data and a generated model that has learned the relationship between pulse wave data measured at an earlobe and the electrocardiographic waveform data, are generated at a time. Two model learning sections 333 (see FIG. 8) are prepared in the model generation device 30.

(3) In the first exemplary embodiment discussed earlier, a generated model is generated by learning the relationship between electrocardiographic waveform data and pulse wave data measured from a single test subject. However, a generated model may be generated by learning the relationship between electrocardiographic waveform data and pulse wave data measured from a plurality of test subjects.

(4) In the embodiments above, the term “processor” refers to hardware in a broad sense. Examples of the processor include general processors (e.g., CPU: Central Processing Unit) and dedicated processors (e.g., GPU: Graphics Processing Unit, ASIC: Application Specific Integrated Circuit, FPGA: Field Programmable Gate Array, and programmable logic device).

In the embodiments above, the term “processor” is broad enough to encompass one processor or plural processors in collaboration which are located physically apart from each other but may work cooperatively. The order of operations of the processor is not limited to one described in the embodiments above, and may be changed.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
a processor configured to estimate a waveform of heartbeats by inputting a waveform of a measured pulse wave to a model constructed by mounting a pulse wave measurement device and a heartbeat measurement device to a test subject and calculating a relationship between respective waveforms output from the devices.

2. The information processing apparatus according to claim 1,
wherein the model is generated by a generative adversarial network, and outputs the waveform of the heartbeats corresponding to the waveform of the pulse wave.

3. The information processing apparatus according to claim 2,
wherein the model is prepared for each location of measurement.

4. The information processing apparatus according to claim 2,
wherein the model is prepared for each user, the waveform of the heartbeats of whom is to be estimated.

5. The information processing apparatus according to claim 2,
wherein the model is generated using the waveform of the pulse wave after being subjected to a correction of a deviation, in a time axis direction, from the waveform of the heartbeats.

6. The information processing apparatus according to claim 5,
wherein the deviation in the time axis direction is given by an amount of shift with which a coefficient of correlation between the waveform of the heartbeats and the waveform of the pulse wave measured concurrently is maximized.

7. The information processing apparatus according to claim 2,
wherein the processor is configured to estimate a peak interval as the waveform of the heartbeats.

8. The information processing apparatus according to claim 1,
wherein the processor is configured to estimate the waveform of the heartbeats by detecting an abnormality in peak interval of the waveform of the measured pulse wave and inputting the waveform of the measured pulse wave to the model which is constructed using a waveform from which the detected abnormality has been excluded.

9. The information processing apparatus according to claim 8,
wherein the processor is configured to estimate the waveform of the heartbeats by inputting the waveform of the measured pulse wave to the model which is constructed using a waveform obtained by subjecting the waveform of the pulse wave from which the abnormality has been excluded to a correction of a deviation, in a time axis direction, from the waveform of the heartbeats.

10. The information processing apparatus according to claim 9,
wherein the processor is configured to estimate the waveform of the heartbeats by inputting the waveform of the measured pulse wave to the model which is constructed by computing a relationship between the waveform of the pulse wave after being subjected to the correction and the waveform of the heartbeats.

11. The information processing apparatus according to claim 8,
wherein the processor is configured to estimate the waveform of the heartbeats by inputting the waveform of the measured pulse wave to the model which is constructed by computing a relationship between the waveform of the pulse wave which is estimated from the waveform from which the abnormality has been excluded and the waveform of the heartbeats.

12. A non-transitory computer readable medium storing a program causing a computer to execute a process comprising:
estimating a waveform of heartbeats by inputting a waveform of a measured pulse wave to a model constructed by mounting a pulse wave measurement device and a heartbeat measurement device to a test subject and calculating a relationship between respective waveforms output from the devices.

13. An information processing apparatus comprising
means for estimating a waveform of heartbeats by inputting a waveform of a measured pulse wave to a model constructed by mounting a pulse wave measurement device and a heartbeat measurement device to a test subject and calculating a relationship between respective waveforms output from the devices.
